(19) United States
(12) Patent Application Publication
(10) Pub. No.: US 2025/0319065 A1
(43) Pub. Date: Oct. 16, 2025

(54) DIMETHYLTRYPTAMINE AND ITS ANALOGS FOR THE PREVENTION, TREATMENT AND RECOVERY OF THE DISORDERS CAUSED BY MYOCARDIAL ISCHEMIA

(71) Applicant: Babywatch, Inc., San Francisco, CA (US)

(72) Inventors: Ede FRECSKA, Debrecen (HU); Josipa VLAINIC, Sesvete (HR); Sandro MUR, Samobor (HR)

(21) Appl. No.: 19/177,065

(22) Filed: Apr. 11, 2025

Related U.S. Application Data
(60) Provisional application No. 63/633,266, filed on Apr. 12, 2024.

(51) Int. Cl. A61K 31/4045; A61K 9/00; A61K 47/02; A61K 47/26

(52) U.S. Cl. CPC A61K 31/4045; A61K 9/0019; A61K 47/02; A61K 47/26

(57) ABSTRACT

Aspects of the invention can include a method of preventing and treating disorders caused by myocardial ischemia (obstructed blood flow to the heart muscle), and a method of recovering from the same by administering an effective amount of a pharmaceutically acceptable composition of N,N-Dimethyltryptamine ((DIMETHYLTRYPTAMINE)) or its analogs thereof. Aspects of the invention can also include a method of preventing and treating myocardial ischemic reperfusion injuries during conditions resulting from inadequate blood flow to the heart muscle, and a method of recovering from the same by administering an effective amount of a pharmaceutically acceptable composition of N,N-Dimethyltryptamine ((DIMETHYLTRYPTAMINE)) or an analog thereof.

### FIELD OF THE INVENTION

Aspects of the invention are related to the field of treating disorders caused by, and methods of recovering from, myocardial ischemia.

### BACKGROUND OF THE INVENTION

Myocardial ischemia, resulting from inadequate blood flow to the heart muscle, underlies a spectrum of cardiovascular conditions, including acute myocardial infarction (AMI), coronary artery disease (CAD), cardiac surgical procedures and various ischemic heart diseases. Myocardial ischemia-reperfusion injury (MIRI) exacerbates tissue damage and dysfunction, posing a significant clinical challenge with substantial morbidity and mortality across these conditions.

In 2020, it is estimated that 244 million people were living with ischemic heart disease (ID) and it was more prevalent in males then in females (141 and 103 million, respectively). Mortality rates were 112 per 100.000. Ischemic heart disease (ID), also called coronary artery disease (CAD), myocardial ischemia, or simply heart disease makes up 15.6% of all deaths worldwide, making it the most common cause of death globally.

Considering the above-presented numbers, the development of novel therapeutic agents to prevent, treat and stimulate recovery after myocardial ischemia occurrence represents a critical area of research in cardiovascular medicine. Surprisingly, it was discovered that (DIMETHYLTRYPTAMINE) and its analogs have beneficial effects on the prevention and treatment of conditions caused by myocardial ischemia, and on promoting the recovery from it, as described in this invention.

DIMETHYLTRYPTAMINE) is endogenously produced in humans and animals (Dean et al., 2019). The first step in (DIMETHYLTRYPTAMINE) biosynthesis is decarboxylation of essential amino acid L-tryptophan by an enzyme called aromatic-L-amino acid decarboxylase (AADC) and conversion to tryptamine. Subsequently, tryptamine is converted via two-step transmethylation to N,N tryptamine ((DIMETHYLTRYPTAMINE)) by the family of enzymes called indole-N-methyl transferase (INMT) and a methyl donor cofactor named S-adenosylhomocysteine (Rosengarten and Friedhoff, 1976).

In vivo and in vitro studies have demonstrated the biosynthesis of (DIMETHYLTRYPTAMINE) in mammalian tissues, specifically the brain tissue. (DIMETHYLTRYPTAMINE) has also been found in the lung tissue, heart tissue, renal tissue and other peripheral tissue and is considered as an endogenous trace neurotransmitter with different physiological roles, including signaling and systemic immunological actions. It is also present in human blood, urine and cerebrospinal fluid (Morales-Garcia et al., 2020).

In addition to the well documented psychedelics effect, (DIMETHYLTRYPTAMINE) has other effects described, including anti-depressant (Cameron et al., 2018), and anti-hypoxic (Szabo et al., 2016) effects. Additionally, it was shown that (DIMETHYLTRYPTAMINE) (in a form of pamoate or nicotinate salt) may be used to treat the stroke and traumatic brain injury (WO-2022160056-A1). One of the recent discovered (DIMETHYLTRYPTAMINE) functions is promotion of structural and functional neural plasticity, and its role in neurogenesis (Ly et al., 2018). Furthermore, it is demonstrated in the literature that it may have anti-inflammatory effect and may contribute to the tissue regeneration (Szabo et al., 2014).

It was shown that (DIMETHYLTRYPTAMINE) binds as an agonist to the serotonergic receptors. Additionally, (DIMETHYLTRYPTAMINE) binds to other receptors from other families. (DIMETHYLTRYPTAMINE) was shown experimentally to bind non-selectively to many of the 5-hydroxytriptamine (5-HT) receptors. 5-HT receptors, or serotonin receptors, are complex receptors found in the central and peripheral nervous system and tissues. Currently, it is experimentally demonstrated that seven types of 5-HT receptors (5-HT₁₋₇) are present in the human body. These seven types of 5-HT receptors are divided into 14 subtypes. All but three subtypes (5-HT₁ₑ, 5-HT_{2C}, 5-HT₆) are expressed in both brain and peripheral tissues (Sharp and Barnes, 2020). According to the literature, (DIMETHYLTRYPTAMINE) binds to 5-HT_{1A}, 5-HT_{1B}, 5-HT_{1D}, 5-HT_{2A}, 5-HT_{2B}, HT_{2C}, 5-HT₆ and 5-HT₇ receptors. (DIMETHYLTRYPTAMINE) binds with the highest affinity to 5-HT_{1A}, 5-HT_{2A}, and 5-HT_{2C} (Strassman, 1994). (DIMETHYLTRYPTAMINE) binding on other 5-HT receptors is yet to be determined. Additionally, (DIMETHYLTRYPTAMINE) shows binding affinity to other receptors as well including dopamine D₁, α1-adrenergic, α2-adrenergic, imidazoline-1, and al receptors as well as serotonin transporter (SERT) (Deliganis et al., 1991; Pierce and Peroutka, 1989; Ray, 2010).

The 5-HT signaling pathway has a major role in the cerebrovascular system and in control of the cardiovascular reflexes, cardiovascular health and disease. For example, 5-HT heart signaling causes a positive chronotropic heart effect and it influences heart arrythmias, atrial fibrillation, coronary artery disease, vasospastic angina (Neumann et al., 2023). Furthermore, T102C polymorphism in 5-HT_{2A}, one of the receptors with a high affinity to (DIMETHYLTRYPTAMINE), is associated with acute myocardial infarction (Doggrell, 2003).

(DIMETHYLTRYPTAMINE) has also shown a significant protective effect in both hypoxia and modulation of innate and adaptive inflammatory responses via Sigma-1 receptor activation (Szabo et al., 2016).

As stated above (DIMETHYLTRYPTAMINE), administered in pharmaceutically acceptable formulations, demonstrates an unexpected effect in prevention, treatment, and recovery of myocardial ischemic events.

Myocardial ischemia (MI) is caused by insufficient blood flow to the heart muscle and can lead to various cardiac conditions. Such conditions include, but are not restricted to, angina pectoris (chest pain or discomfort due to reduced blood flow to the heart muscle) including prinzmetal angina or variant angina (episodes of chest pain caused by temporary coronary artery spasms, leading to reduced blood flow) and microvascular angina (chest pain resulting from dysfunction or abnormalities in the smaller blood vessels of the heart); acute coronary syndrome (ACS, includes unstable angina and myocardial infarction or heart attack), coronary artery disease (CAD, buildup of plaque in the coronary arteries restricting blood flow to the heart), myocardial infarction (MI, commonly known as a heart attack, it results from prolonged ischemia leading to damage or death of heart tissue); silent ischemia (ischemia without noticeable symptoms, but still poses a risk of heart damage); chronic ischemic heart disease (long-term reduced blood supply to the heart, often associated with CAD); ischemic cardiomyopathy (long-term myocardial ischemia can contribute to the development of cardiomyopathy, a condition where the heart muscle weakens, affecting its pumping ability); ventricular fibrillation. In extreme cases of myocardial ischemia, irregular heart rhythms like ventricular fibrillation can occur, posing a life-threatening emergency.

Myocardial ischemia reperfusion injury (MIRI) is a complex phenomenon that starts with an ischemic event, i.e., when the blood flow to the heart muscle or myocardium is temporarily restricted. If the blood flow to the heart is restored (reperfusion) the tissue injury will occur. Myocardial ischemia reperfusion injury is commonly seen during events such as heart attacks or during surgical procedures like coronary artery bypass grafting (CABG) or percutaneous coronary intervention (PCI). During tissue ischemia, the lack of oxygen and nutrients can lead to cellular damage and dysfunction within the heart tissue. However, the restoration of blood flow, while essential for tissue survival, paradoxically exacerbates the injury through a cascade of biochemical and cellular events, resulting in myocardial reperfusion injury. Therefore, myocardial ischemia reperfusion injury poses a significant challenge in the management of cardiovascular diseases where development of effective therapeutic strategies to mitigate its impact on patient outcomes is of critical importance. Myocardial ischemia-reperfusion injury can occur in various clinical scenarios where blood flow to the heart muscle is temporarily restricted and then restored. Clinical scenarios include but are not limited to acute myocardial infarction (heart attack), coronary artery bypass grafting (CABG), percutaneous coronary intervention (PCI) coronary artery spasm, coronary microvascular dysfunction, cardiac arrest and resuscitation and the organ transplantation.

### SUMMARY OF THE INVENTION

Aspects of the invention include a method for treating cardiovascular conditions associated with inadequate myocardial perfusion, the method including: administering (DIMETHYLTRYPTAMINE), or an analog compound thereof, in therapeutically effective amounts, wherein the (DIMETHYLTRYPTAMINE) or an analog compound thereof is selected from one or more of: a (DIMETHYLTRYPTAMINE) base molecule and/or its pharmaceutically acceptable salts and analogs thereof; a 5-MeO-(DIMETHYLTRYPTAMINE) base or its salts; isotopically labeled (DIMETHYLTRYPTAMINE), or analogs thereof.

Aspects of the invention can also include a pharmaceutical composition for the treatment of cardiovascular conditions associated with inadequate myocardial perfusion, including: an effective amount of (DIMETHYLTRYPTAMINE) or (DIMETHYLTRYPTAMINE) analogs, wherein the (DIMETHYLTRYPTAMINE) or (DIMETHYLTRYPTAMINE) analogs are in a formulation including one or more of, a diluent; organic solvents; preservatives; buffers; tonicity agents; solubilizers; pH adjusting agents; osmolytes; or chelating agents.

### DETAILED DESCRIPTION

It should be noted that the invention is not limited examples provided herein, which are referred to for purposes of illustration only.

In this regard, in the descriptions herein, certain specific details are set forth in order to provide a thorough understanding of various embodiments or aspects of the invention. However, one skilled in the art will understand that the invention may be practiced at a more general level without one or more of these details.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, characteristics of different embodiments may be combined in any suitable manner to form one or more other embodiments. In addition, the terms “aspect” or “aspects” can be used interchangeably with the term embodiment or embodiments.

(DIMETHYLTRYPTAMINE), administered in pharmaceutically acceptable formulations, demonstrates an unexpected effect in prevention, treatment, and recovery of myocardial ischemic events, thereby providing a new method for therapeutic intervention.

It was surprisingly found that (DIMETHYLTRYPTAMINE) and its analogs exert their therapeutic effects through a unique mechanism of action, targeting serotonergic receptors, which play a central role in the pathophysiology of myocardial ischemia. It was surprisingly demonstrated that by intracellular response, (DIMETHYLTRYPTAMINE) mitigates myocardial ischemic injury and improves cardiac function especially following ischemia-reperfusion events.

The invention teaches the use of (DIMETHYLTRYPTAMINE) or its analogs for the prevention and treatment of myocardial ischemia related conditions and promoting the recovery from it.

The invention teaches the use of (DIMETHYLTRYPTAMINE) or its analogs for the prevention and treatment of myocardial ischemia reperfusion injury related conditions and promoting the recovery from it.

The invention teaches a method for prevention and treatment of myocardial ischemia related conditions, and promoting the recovery from it, comprising administration of (DIMETHYLTRYPTAMINE) or its analogs.

The invention teaches a method for prevention and treatment of myocardial ischemia reperfusion injury related conditions, and promoting the recovery from it, comprising administering (DIMETHYLTRYPTAMINE) or its analogs.

In another embodiment, the invention teaches the usage of a pharmaceutically acceptable salt of (DIMETHYLTRYPTAMINE) or its analogs to treat myocardial ischemia related conditions and myocardial ischemia reperfusion injury related conditions.

In this disclosure, reference is made to a number of terms, which are to be understood to have the meanings as below unless stated differently.

Compounds nomenclature mentioned herein are intended to be in agreement with the International Union of Pure and Applied Chemistry (IUPAC), precisely “IUPAC Compendium of Chemical Terminology (Gold Book)” (A. D. Jenkins et al., Pure & Appl. Chem., 1996, 68, 2287-2311).

As used herein, the use of singular nouns is intended to encompass their respective plural counterparts and vice versa, unless stated differently.

As used herein, the term “patient” or “subject” can be used interchangeably and refers to a mammal. Generally, the mammal is a human, but the term mammal may refer to any other mammal.

As used herein, the term “treatment” defines interventions aimed at a patient for the purpose of reducing disorder progression, achieving a cure, or implementing prophylaxis to prevent the onset or recurrence of a disorder where prophylaxis aims to decrease the risk of developing a disorder, but it may not entirely eliminate the possibility of the disease occurrence. Typically, treatment is not prophylactic but rather is administered to a patient having a diagnosed or suspected disorder.

As used herein, the term “prevention” refers to actions or measures taken to avoid the occurrence, onset, or progression of a targeted disease, injury, or other adverse health condition. The goal of prevention is to reduce the burden of illness and promote overall health and well-being by minimizing risk factors and addressing underlying causes.

As used herein, the term “recovery” refers to the process of regaining health, wellness, or normal functioning after experiencing illness, injury, or adversity.

As used herein, the term “analog” or “analog compound” herein defines chemical compounds that have similar physical, chemical, biochemical, or pharmacological properties.

As used herein, the term “analog” defines either a (DIMETHYLTRYPTAMINE) base molecule either pharmaceutically acceptable salts thereof or any mixture thereof. The term “analog” also includes but is not restricted to the 5-MeO-(DIMETHYLTRYPTAMINE) base or any pharmaceutically acceptable salts or any mixture thereof. The term “analog” also includes any isotopically labelled compound of the (DIMETHYLTRYPTAMINE) and 5-MeO-(DIMETHYLTRYPTAMINE) bases, pharmaceutically acceptable salts or analogs, including any mixture thereof.

As used herein, the term “pharmaceutically acceptable” refers to a formulation that is composed of the active pharmaceutical ingredient such as (DIMETHYLTRYPTAMINE) or its analogs mixed with an excipient, such as diluent (e.g., Water For Injection, 5% Dextrose, 0.9% Sodium Chloride), organic solvents, preservatives, buffers, tonicity agents, solubilizers, pH adjusting agents, osmolytes, chelating agents, or excipients with any other function which does not affects the biological activity or properties of the active pharmaceutical ingredient. Such pharmaceutically acceptable formulation is non-toxic in the amounts administered.

As used herein, “pharmaceutically acceptable salt” refers to a salt that is safe for use in pharmaceutical formulations and does not introduce any harmful effects to the patient when administered according to prescribed dosage regimens. Such a salt may be prepared from active ingredient bases, using inorganic acids, inorganic bases, organic acids, organic bases, solvates, hydrates, and clathrates or any mixture thereof.

As used herein, a “pharmaceutically effective amount,” “therapeutically effective amount,” or “effective amount” of a compound is that amount of compound that is sufficient to provide beneficial effect to the subject to which the compound is administered.

Aspects of the invention are related to pharmacological mechanisms by which (DIMETHYLTRYPTAMINE) exerts effects on myocardial ischemia and myocardial ischemia reperfusion injury.

Aspects of the invention are further related to optimal dosages, routes of administration, and potential combination therapies.

Aspects of the present invention provide a novel and potent therapeutic approach related to the use of N,N-Dimethyltryptamine ((DIMETHYLTRYPTAMINE)) or its pharmaceutically acceptable salts for the treatment of a broad spectrum of cardiovascular conditions associated with inadequate myocardial perfusion. (DIMETHYLTRYPTAMINE), known for its psychoactive properties, has been unexpectedly discovered to exhibit therapeutic effects in improving overall cardiovascular health, preventing or reducing ischemic events, and addressing conditions related to atherosclerosis, thrombosis, vasospasm, and coronary artery disease.

In view of the present disclosure, optimal dosages of (DIMETHYLTRYPTAMINE) or its analogs for treating cardiovascular conditions may vary depending on the specific indication and patient status. An appropriate amount of (DIMETHYLTRYPTAMINE) or its analogs can be administered orally, intravenously, intramuscularly, intranasally, or by any other suitable route.

Furthermore, in aspects of the present invention, (DIMETHYLTRYPTAMINE) or its analogs can be used in combination with other therapeutic agents for cardiovascular diseases. Such combinations may include, but are not limited to, antiplatelet agents, anticoagulants, beta-blockers, calcium channel blockers, vasodilators, and other agents known for their cardiovascular benefits. These combinations aim to synergistically enhance the therapeutic effects and provide a comprehensive approach to cardiovascular care.

Aspects of the invention encompass preventive measures for individuals at risk, interventions for specific cardiovascular disorders, and strategies for promoting overall cardiovascular health.

Aspects of the invention can include combinations of one or more of the following features.

A method for effectively treating cardiovascular conditions associated with inadequate myocardial perfusion, including myocardial ischemia in patients, comprising administering (DIMETHYLTRYPTAMINE) or its analog compounds in therapeutically effective amounts. The (DIMETHYLTRYPTAMINE) or analog compound is selected from (DIMETHYLTRYPTAMINE) base molecule and/or its pharmaceutically acceptable salts, analogs thereof, 5-MeO-(DIMETHYLTRYPTAMINE) base or its salts, isotopically labeled (DIMETHYLTRYPTAMINE), analogs of the analogs, or any suitable mixture thereof.

Aspects of the invention include a method wherein the treatment may occur before, during and after myocardial perfusion event.

Aspects of the invention include a method wherein different cardiovascular conditions are treated in a patient in need as a result of a myocardial ischemia.

Aspects of the invention include a method wherein the treatment comprises preventing or reducing ischemic events in individuals at risk of or diagnosed with cardiovascular conditions associated with inadequate myocardial perfusion.

Aspects of the invention include a method wherein cardiovascular conditions include conditions related to angina pectoris including prinzmetal or variant angina and microvascular angina.

Aspects of the invention include a method wherein cardiovascular conditions include conditions related to acute coronary syndrome (ACS), including unstable angina and myocardial infarction.

Aspects of the invention include a method wherein cardiovascular conditions include conditions related to thrombosis, vasospasm, coronary artery disease (CAD) and myocardial Infarction (MI).

Aspects of the invention include a method wherein cardiovascular conditions include conditions related to silent ischemia.

Aspects of the invention include a method wherein cardiovascular conditions include conditions related to chronic ischemic heart disease.

Aspects of the invention include a method wherein cardiovascular conditions include conditions related to ischemic cardiomyopathy.

Aspects of the invention include a method wherein cardiovascular conditions include conditions related to ventricular fibrillation as result of myocardial ischemia.

Aspects of the invention include a method wherein treatment is included that enhances overall cardiovascular health, promotes vasodilation, reduces inflammation, and improves endothelial function.

Aspects of the invention include a method that including treating patients for myocardial ischemia reperfusion injury (MIRI) during various clinical scenarios and cardiovascular conditions.

Aspects of the invention include a method that includes treating patients for myocardial ischemia-reperfusion injury in various clinical scenarios and cardiovascular conditions, including those occurring before, during, or after acute myocardial infarction

Aspects of the invention include a method that includes treating patients for myocardial ischemia-reperfusion injury in various clinical scenarios and cardiovascular conditions, including those occurring before, during, or after Coronary Artery Bypass Grafting (CABG)

Aspects of the invention include a method that includes treating patients for myocardial ischemia-reperfusion injury in various clinical scenarios and cardiovascular conditions, including those occurring before, during, or after Percutaneous Coronary Intervention (PCI).

Aspects of the invention include a method that includes treating patients for myocardial ischemia-reperfusion injury in various clinical scenarios and cardiovascular conditions, including those occurring before, during, or after Coronary Artery Spasm.

Aspects of the invention include a method that includes treating patients for myocardial ischemia-reperfusion injury in various clinical scenarios and cardiovascular conditions, including those occurring before, during, or after Coronary Microvascular Dysfunction.

Aspects of the invention include a method that includes treating patients for myocardial ischemia-reperfusion injury in various clinical scenarios and cardiovascular conditions, including those occurring before, during, or after Cardiac Arrest and Resuscitation.

Aspects of the invention include a method that includes treating patients for myocardial ischemia-reperfusion injury before, during, or after heart transplantation

Aspects of the invention also include a method that includes intravenous administration of the (DIMETHYLTRYPTAMINE) or its analogs at the appropriate amount.

Aspects of the invention also include a method that includes intramuscular administration of the (DIMETHYLTRYPTAMINE) or its analogs at an appropriate amount.

Aspects of the invention also include a method that includes oral administration of the (DIMETHYLTRYPTAMINE) or its analogs at an appropriate amount.

Aspects of the invention also include a method that includes intranasal administration of the (DIMETHYLTRYPTAMINE) or its analogs at an appropriate amount.

Aspects of the invention also include a method wherein the administering step comprises of administering 1-200 mg of compound intravenously or by intramuscular injection.

Aspects of the invention also include a pharmaceutical composition for the treatment of cardiovascular conditions associated with inadequate myocardial perfusion, comprising an effective amount of (DIMETHYLTRYPTAMINE) or (DIMETHYLTRYPTAMINE) analogs in a pharmaceutically acceptable formulation containing diluent (e.g., Water For Injection, 5% Dextrose, 0.9% Sodium Chloride), organic solvents, preservatives, buffers, tonicity agents, solubilizers, pH adjusting agents, osmolytes, or chelating agents.

Aspects of the invention also include a feature wherein the cardiovascular conditions including but not limited to inadequate myocardial perfusion, angina pectoris, prinzmetal or variant angina, microvascular angina, acute coronary syndrome, thrombosis, vasospasm, coronary artery disease, myocardial Infarction, silent ischemia, chronic ischemic heart disease, ischemic cardiomyopathy, ventricular fibrillation as result of myocardial ischemia.

Aspects of the invention also include a feature wherein the method is for promoting and improving overall cardiovascular health, including promoting vasodilation, reducing inflammation, and enhancing endothelial function

Aspects of the invention also include a feature wherein the administration is performed prophylactically to individuals with predisposing factors for cardiovascular conditions associated with inadequate myocardial perfusion.

Aspects of the invention also include a feature wherein the cardiovascular conditions including but not limited to inadequate myocardial perfusion, angina pectoris, prinzmetal or variant angina, microvascular angina, acute coronary syndrome, thrombosis, vasospasm, coronary artery disease, myocardial Infarction, silent ischemia, chronic ischemic heart disease, ischemic cardiomyopathy, ventricular fibrillation as result of myocardial ischemia.

Aspects of the invention also include a method for reducing the risk of myocardial infarction in a subject at risk thereof, comprising administering an effective amount of the DMR analogs from feature 1 to said subject.

Aspects of the invention also include a feature wherein said subject at risk comprises individuals with a history of cardiovascular conditions including but not limited to inadequate myocardial perfusion, angina pectoris, prinzmetal or variant angina, microvascular angina, acute coronary syndrome, thrombosis, vasospasm, coronary artery disease, myocardial Infarction, silent ischemia, chronic ischemic heart disease, ischemic cardiomyopathy, ventricular fibrillation as result of myocardial ischemia.

It should be obvious to one of ordinary skill in the art that subsets or combinations of various embodiments described above provide further embodiments.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

What is claimed is:

1. A method for treating cardiovascular conditions associated with inadequate myocardial perfusion, the method comprising:
administering (DIMETHYLTRYPTAMINE), or an analog compound thereof, in therapeutically effective amounts,
wherein the (DIMETHYLTRYPTAMINE) or an analog compound thereof is selected from one or more of:
a (DIMETHYLTRYPTAMINE) base molecule and/or its pharmaceutically acceptable salts and analogs thereof;
a 5-MeO-(DIMETHYLTRYPTAMINE) base or its salts;
isotopically labeled (DIMETHYLTRYPTAMINE), or analogs thereof.

2. The method of claim 1, wherein the administering comprises administering 1-200 mg of the (DIMETHYLTRYPTAMINE), or the analog compound thereof intravenously or by intramuscular injection.

3. The method of claim 1, wherein the administering comprises administering the (DIMETHYLTRYPTAMINE), or the analog compound thereof at an appropriate amount.

4. The method of claim 1, wherein the administering comprises intravenous administration of the (DIMETHYLTRYPTAMINE), or the analog compound thereof at an appropriate amount.

5. The method of claim 1, wherein the administering comprises intramuscular injection administration of the (DIMETHYLTRYPTAMINE), or the analog compound thereof at an appropriate amount.

6. The method of claim 1, wherein the administering comprises oral administration of the (DIMETHYLTRYPTAMINE), or the analog compound thereof at an appropriate amount.

7. The method of claim 1, wherein the administering comprises intramuscular injection administration of the (DIMETHYLTRYPTAMINE), or the analog compound thereof before, during and after a myocardial perfusion event.

8. The method of claim 1, wherein the administering comprises prophylactic administering to individuals with predisposing factors for cardiovascular conditions associated with inadequate myocardial perfusion.

9. The method of claim 1, wherein the method includes treating patients for myocardial ischemia-reperfusion injury in various clinical scenarios and cardiovascular conditions, including those occurring before, during, or after acute myocardial infarction.

10. The method of claim 1, wherein the administering comprises administration of the (DIMETHYLTRYPTAMINE) before, during and after a myocardial perfusion event.

11. The method of claim 1, wherein the administering comprises administration the analog compound before, during and after a myocardial perfusion event.

12. The method of claim 1, wherein the method includes treating patients having different cardiovascular conditions as a result of a myocardial ischemia.

13. The method of claim 1, wherein the treatment comprises preventing or reducing ischemic events in individuals at risk of or diagnosed with cardiovascular conditions associated with inadequate myocardial perfusion.

14. The method of claim 1, wherein the method includes treating patients, wherein the cardiovascular conditions include conditions related to angina pectoris including prinzmetal or variant angina and microvascular angina.

15. The method of claim 1, wherein the method includes treating patients, wherein cardiovascular conditions include conditions related to acute coronary syndrome (ACS), including unstable angina and myocardial infarction.

16. The method of claim 1, wherein the method includes treating patients, wherein cardiovascular conditions include conditions related to thrombosis, vasospasm, coronary artery disease (CAD) and myocardial Infarction (MI).

17. A pharmaceutical composition for the treatment of cardiovascular conditions associated with inadequate myocardial perfusion, comprising:
an effective amount of (DIMETHYLTRYPTAMINE) or (DIMETHYLTRYPTAMINE) analogs,
wherein the (DIMETHYLTRYPTAMINE) or (DIMETHYLTRYPTAMINE) analogs are in a formulation including one or more of,
a diluent;
organic solvents;
preservatives;
buffers;
tonicity agents;
solubilizers;
pH adjusting agents;
osmolytes; or
chelating agents.

18. The pharmaceutical composition of claim 17, wherein
the diluent includes
water for injection,
Dextrose at 5%; and
Sodium Chloride at 0.9%.
